(19) United States
(12) Patent Application Publication
(10) Pub. No.: US 2026/0341429 A1
(43) Pub. Date: Oct. 29, 2026

(54) SYSTEMS AND METHODS FOR MEDICATION AND DISEASE TRACKING

(71) Applicant: Sanofi, Paris (FR)

(72) Inventors: Niranjan Tumminkatti, Cambridge, MA (US); Jesse Pete Alvarez, Cambridge, MA (US); Marjorie Beaugad, Paris (FR); Véronique Bruhat, Paris (FR); John Choquette, Cambridge, MA (US); Caroline Lanzoni McCray, Cambridge, MA (US); Junqian Liang, Cambridge, MA (US)

(21) Appl. No.: 19/123,277

(22) PCT Filed: Oct. 24, 2023
(86) PCT No.: PCT/IB2023/000625

§ 371 Date: Apr. 22, 2025

Related U.S. Application Data
(60) Provisional application No. 63/418,738, filed on Oct. 24, 2022.

(30) Foreign Application Priority Data
Mar. 31, 2023 (EP) 23315073.9

(51) Int. Cl. G16H 10/60; A61B 5/00; G01D 21/02; G06V 20/50; G16H 50/30

(52) U.S. Cl. CPC G16H 10/60; A61B 5/4866; A61B 5/7246; A61B 5/7275; A61B 5/742; G01D 21/02; G06V 20/50; G16H 50/30; A61B 2560/0242; G06V 2201/03

(57) ABSTRACT

The present specification relates to systems and methods for medication and disease tracking management and in particular to a computer implemented method for using environment and/or user specific health information to trigger a prompt to make a journal entry. According to a first aspect of this specification, there is described a non-transitory computer readable storage medium comprising instructions that, when executed by a computer, cause the computer to: execute a program for tracking a user's medical condition; obtain environmental information or user specific health information; determine that a trigger condition is met based on the obtained environmental information or user specific health information; and in response to determining that the trigger condition is met, generate a notification to be displayed on a user device prompting the user to input data into a medical journal within the program.

### CROSS REFERENCE TO RELATED APPLICATIONS

This application is the U.S. National Phase Application under 35 U.S.C. § 371 of International Patent Application No. PCT/IB2023/000625, filed on Oct. 24, 2023, which claims the benefit of European Patent Application No. 23315073.9, filed on Mar. 31, 2023 and which also claims the benefit of U.S. Patent Application No. 63/418,738, filed on Oct. 24, 2022. The entire contents of the foregoing are hereby incorporated by reference.

### TECHNICAL FIELD

The present specification relates to systems and methods for medication and disease tracking management and in particular to a computer implemented system and method for using environment and/or user specific health information to trigger a prompt to make a journal entry.

### BACKGROUND

A significant barrier to high quality therapeutic outcomes is patient engagement and adherence with a prescribed medication regimen as well as patient understanding of the factors affecting their disease progression and overall health.

Current systems do not effectively engage users or provide appropriate and timely messaging and reminders. Current systems do not provide sufficient opportunity for a user to input information relating to the symptoms and treatment in a timely fashion and do not sufficiently leverage this information to make informed treatment and lifestyle decisions.

### SUMMARY

Certain aspects of this disclosure relate to an effective symptom and treatment journaling system, which can encourage patient engagement and adherence and ultimately lead to improved therapeutic outcomes for the patient.

A first aspect of this disclosure provides non-transitory computer readable storage medium comprising instructions that, when executed by a computer, cause the computer to:
execute a program for tracking a user's medical condition;
obtain environmental information or user specific health information, wherein the obtained environmental information or user specific health information is indicative of a possible exacerbation of the user's medical condition;
determine that a trigger condition is met based on the obtained environmental information or user specific health information; and
in response to determining that the trigger condition is met, generate a notification to be displayed on a user device prompting the user to input data into a medical journal within the program.

In some embodiments, the program is configured to track a plurality of different medical conditions of the user. The computer can be part of a user device and the instructions may be further configured to cause the computer to obtain the environmental information using one or more sensors forming part of the user device.

The sensors can comprise one or more of a thermometer, a barometer and/or a hygrometer. Alternatively, or in addition, the computer can obtain the environmental information from a weather application running on the user device. The environmental information can comprise one or more of: a temperature; a humidity; an air pressure; a pollen count; a pollution index; an NO₂ count; an air quality index; a UV index; and/or a weather condition or weather prediction.

The trigger condition can comprise one or more of: an upper temperature threshold; a lower temperature threshold; an upper humidity threshold; an upper pollen count threshold; an upper NO₂ count threshold; a threshold air quality index score; and/or one or more sets of predefined weather conditions.

The computer can obtain the user specific health information from one or more sensors of the user device. The one or more sensors can comprise an optical sensor.

The computer can obtain the user specific health information from one or more external devices. The one or more external devices can comprise a wearable device selected from a list comprising, a smart watch, a step meter, a glucose meter and a pulse oximeter. The user specific health information can comprise one or more of: a heart rate, a breathing rate, a temperature, a blood pressure, a blood oxygen level, a number of steps taken in a specific time period, an amount of sleep and/or a sleep pattern.

The trigger condition can comprise one or more of: an upper and/or lower threshold for a heartrate; an upper/lower threshold for a blood pressure, a lower threshold for a blood oxygen level, an upper/lower temperature threshold, an upper/lower breathing rate threshold, a lower threshold for a number of steps taken in a specific time period and/or a deviation from an established or predefined sleep pattern.

The user specific health information can comprise one or more of: calorie intake, fat intake and/or salt intake. The generated notification can provide a warning about the environmental information or the user specific health information that triggered the generation of the notification.

The computer may be part of a user device and the instructions may be further configured to cause the computer to display the generated notification on the user device as a push notification to a locked screen of the user device. The instructions can further cause the computer to create a journal entry within the program comprising information regarding symptoms of the user's medical condition reported by the user.

The information regarding symptoms of the user's medical condition can comprise information regarding symptoms of a plurality of different medical conditions. The instructions can further cause the computer to compare the information regarding symptoms of the user's medical condition reported by the user with contemporary environmental information or user specific health information to identify one or more correlations. Based on the identified one or more correlations, the computer can identify that current environmental information or user specific health information is indicative of a risk factor for the user's medical condition. In response, the computer can generate a notification to be displayed on the user device to alert the user to the current environmental information or user specific health information and/or to the risk factor.

A second aspect of the present disclosure provides a computer implemented method, the method comprising:
executing a program for tracking a user's medical condition;
obtaining environmental information or user specific health information;
determining that a trigger condition is met based on the obtained environmental information or user specific health information; and
   in response to determining that the trigger condition is met, generating a notification to be displayed on a user device prompting the user to input data into a medical journal within the program.

A third aspect of the present disclosure provides a user device comprises processing circuitry configured to generate and display a notification on a display of the user device, the notification prompting the user to input data into a medical journal within the program. The user device can receive a user input selecting one or more health conditions that the journal entry will relate to, display one or more queries regarding a first selected health condition, and after completion of a journal entry relating to the first selected health condition, display an option to complete a questionnaire relating to the first selected health condition.

The one or more queries regarding a first selected health condition can include a request to specify a time and/or location relating to an episode of the first health condition. The one or more queries regarding a first selected health condition can include a request to specify a body location at which the health condition is occurring.

After the completion of the journal entry relating to the first selected health condition, the user device can display one or more queries regarding a second selected health condition. After the completion of the journal entry relating to the first selected health condition, the user device can display an option to set a reminder to make a subsequent journal entry.

A fourth aspect of the present disclosure provides a user device comprises processing circuitry configured to execute a program for tracking a user's medical condition, obtain environmental information or user specific health information, determine that a trigger condition is met based on the obtained environmental information or user specific health information, and in response to determining that the trigger condition is met, generate a notification to be displayed on a user device prompting the user to input data into a medical journal within the program.

A fifth aspect of the present disclosure provides a computer implemented method comprises executing a program for creating journal entries for tracking a user's medical condition, creating a journal entry within the program comprising information regarding symptoms of the user's medical condition reported by the user, associating environmental conditions and/or user specific health information with the journal entry, identifying a risk factor for the user's medical condition by correlating the symptoms of the user's medical condition and the associated environmental conditions and/or user specific health information, and generating a notification to be displayed on a user device to alert the user to the risk factor for the user's medical condition.

The method can further comprise obtaining information regarding new environmental conditions and/or new user specific health information, determining that the new environmental conditions and/or new user specific health information is correlated with an identified risk factor for the user's medical condition, and generating a notification to be displayed on the user device to alert the user to the new environmental information or new user specific health information and/or to the risk factor.

The method can further comprise creating a plurality of journal entries within the program, each journal entry comprising information regarding symptoms of the user's medical condition reported by the user, associating environmental conditions and/or user specific health information with each of the plurality of journal entries, identifying a risk factor for the user's medical condition by correlating the symptoms of the user's medical condition and the associated environmental conditions and/or user specific health information for each of the plurality of journal entries and amalgamating each identified risk factor.

### BRIEF DESCRIPTION OF THE FIGURES

So that the general concepts set out in the foregoing sections can be more fully understood, embodiments thereof will be described with reference to the accompanying drawings, in which:

FIG. 1 shows schematic overview of an overall architecture for a disease management and medication regimen management system;

FIGS. 2a to 2c show three different ways in which the application can prompt the user to make a journal entry;

FIGS. 2d and 2e show an alternative homepage GUI and a journal page GUI;

FIGS. 3a to 3e show an example process for making a selection to create a journal entry relating to Atopic Dermatitis and the steps involved in making that entry;

FIGS. 4a to 4e show an example process for making a selection to create a journal entry relating to Asthma and the steps involved in making that entry;

FIG. 5 shows an example process for making a selection to create a journal entry relating to Overall Health and a single page with the entry options;

FIGS. 6a to 6d show examples of screens showing insights on overall health and environmental data which may be displayed after FIGS. 3d, 4e and/or 5 in their respective processes;

FIGS. 6e and 6f show examples of screens providing options to begin clinically approved questionnaires;

FIG. 7 illustrates schematically a user device according to some embodiments;

FIG. 8 shows a flow chart of an example method for triggering a journal notification; and

FIG. 9 shows a flow chart of an example method for identifying a risk factor for a user's medical condition.

### DETAILED DESCRIPTION

The present disclosure relates to an application which can be run on a user device and which assists a user with tracking of their health and disease progression and management of all aspects of their treatment. In particular, the application encourages and prompts users to make journal entries to document the progress of their treatment (symptoms, flares, mood, sleep, anything related to their condition).

The application can be used to manage a broad range of diseases which are treated by a range of different medicaments. Although some of the specific embodiments below are described in relation to the treatment of atopic dermatitis and/or asthma using a single medicament, the application is not so limited. Various other immunology indications may also be managed by the application, where one or more of these immunology indications may be treated by using a single medicament approved for use in treating the one or more immunology indications. Such a single medicament can include, for example, different dosages containing the same API, different volumes or concentrations of the same API, or different formulations containing the same API. In some embodiments, a single medicament can include an anti IL-4R mAb (e.g., Dupilumab). Further, a broad range of immunological conditions may be managed by the application. The user may be prescribed one or more drugs and the application provides personalized support for users depending on their particular drug prescription(s) and health profile. For example, the application may be configured to provide support for a number of diseases caused by Type 2 inflammation. In the context of dermatology this may include Atopic Dermatitis, Prurigo Nodularis, Bullous Pemphigoid, an Urticaria (e.g., chronic spontaneous urticaria, or cold inducible urticaria), Hand and Foot Disease, or Pruritis, each of which may be treated by injections of Dupixent or other injectable drugs, but also by some oral and topical medications. In the context of respiratory diseases, this may include Asthma, Nasal Polyps, Sinusitis (e.g., chronic sinusitis with or without nasal polyps), Allergic Bronchopulmonary aspergillosis (ABPA), or Allergic Fungal Rhinosinusitis (AFRS) (which may be treated by injections of Dupixent® or other injectable drugs but also by some oral medications) and Chronic Obstructive Pulmonary Disease (COPD), which may be treated by injections of Dupixent® or other injectable drugs. In the context of Gastroenterology this may include Inflammatory Bowel Disease (IBD), Eosinophilic Esophagitis (EoE), Eosinophilic Gastroenteritis (EGE), or Ulcerative Colitis which may be treated by injections of Dupixent® or other injectable drugs but also by some oral medications.

The medicament or medicaments may be administered by injection. As used herein, the term injection or self-injection is intended to encompass intra-venous injection, intra-muscular injection, infusion, or any needle-based injection system as described in Table 1 of section 5.2 of ISO 11608-1: 2014(E). As described in ISO 11608-1: 2014(E), needle-based injection systems may be broadly distinguished into multi-dose container systems and single-dose (with partial or full evacuation) container systems. The container may be a replaceable container or an integrated non-replaceable container.

The application assists the user in managing their disease, such as by providing a tracking journal in which user's and their health care providers can monitor treatment use and progress by logging quick journal entries about symptoms and medicament use and by generating reports based on the collected information. The application mays also encourage and prompt users to maintain their treatment routine through a series of notifications on various aspects of their treatment, e.g. reminders to take medication, reminder about doctor's appointments and to generate reports in advance of these, reminders about calls and to make appropriate preparations for these, reminders about medicament delivery or prescription end. The application can additionally assist the user in accessing treatment, including accessing insurance coverage, managing delivery of medicaments and providing education support. The application may also provide holistic care by providing access to learning material including instructional articles and videos and motivational articles and challenges, and by providing a connection to other connected devices, such as activity trackers and heart rate monitors to continuously update and learn about user's behavior. The application may also gather and combine information from other programs such as environmental monitoring apps or weather forecasts.

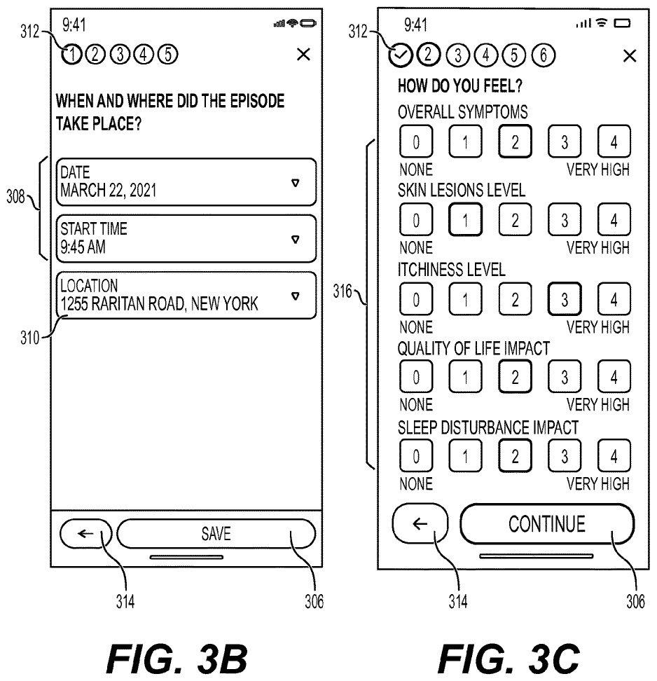

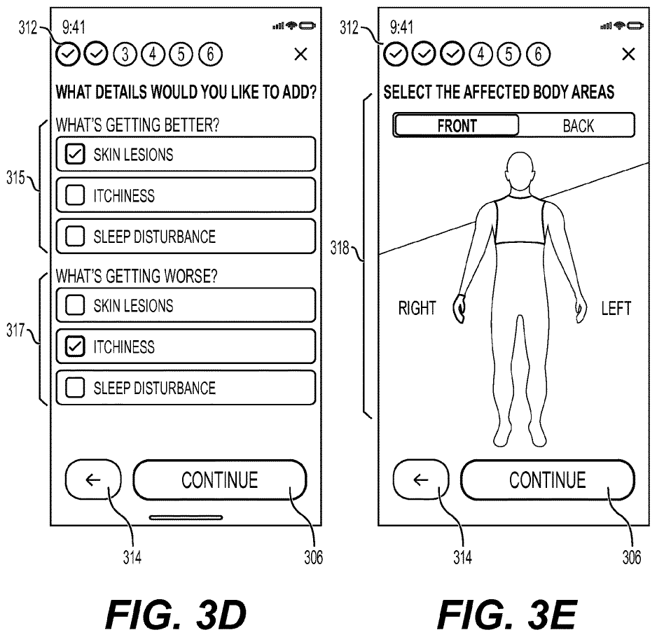

The application allows the user to make journal entries to document the progress of their treatment (symptoms, flares, mood, sleep, anything related to their condition). As described below in relation to FIGS. 3-6, the application may track the user progress by:
(a) Presenting the user with a series of questions based on whether the user indicates they have one or more medical conditions from a set of medical conditions, e.g. whether a user indicates they have disease A or B or both. For example, the set of questions presented to a user who indicates they have disease A may differ from those presented to a user to indicates disease B. The questions may relate to the present symptoms of the user and the severity of those symptoms;
(b) Once the user has answered the series of questions, or at various times while the user is answering the questions, the journal entry is saved. The user may be asked to set a periodic journal reminder (e.g., daily, weekly);
(c) The user may have the facility to make free text entries to the journal; and
(d) Toward the end of the process of creating the journal entry, the application may share some statistics on environmental conditions (e.g., current air quality index in the patient's area) and recent personal health metrics (e.g., average hours of sleep that week, step count) for the user to consider.

The application may further provide smart insights into the medical condition(s) of the user to assist the user in better managing the condition(s), for example by:
(a) Leveraging the journal entries and trigger understanding to anticipate the next flare and manage symptoms. For example, based on a previous journal entry about an episodic flare (exacerbation of an unpleasant symptom) and environmental or other data related to that journal entry (e.g. pollen count or humidity at the time of the flare), the application attempts to predict a flare/episode before it occurs. The application may then notify the user about the predicted flare/episode. The application may suggest steps the user can take to prevent or mitigate the flare/episode.
(b) The application allows/encourages the user to be specific regarding symptoms (e.g. identifying which areas on the body are better/worse). The application may encourage the user to add photos of their skin/rashes or other external symptoms of the condition.

The application may further proactively present relevant information to the patient based on information shared in their journal entries.

The application may further encourage patients to create additional journal entries to record potentially relevant events/health changes. For example, the application may access the fitness data of the patient from another application and note that the patient hasn't been sleeping well. The application then encourages the patient to start a journal entry to make note of this change and provide any additional information that may be relevant.

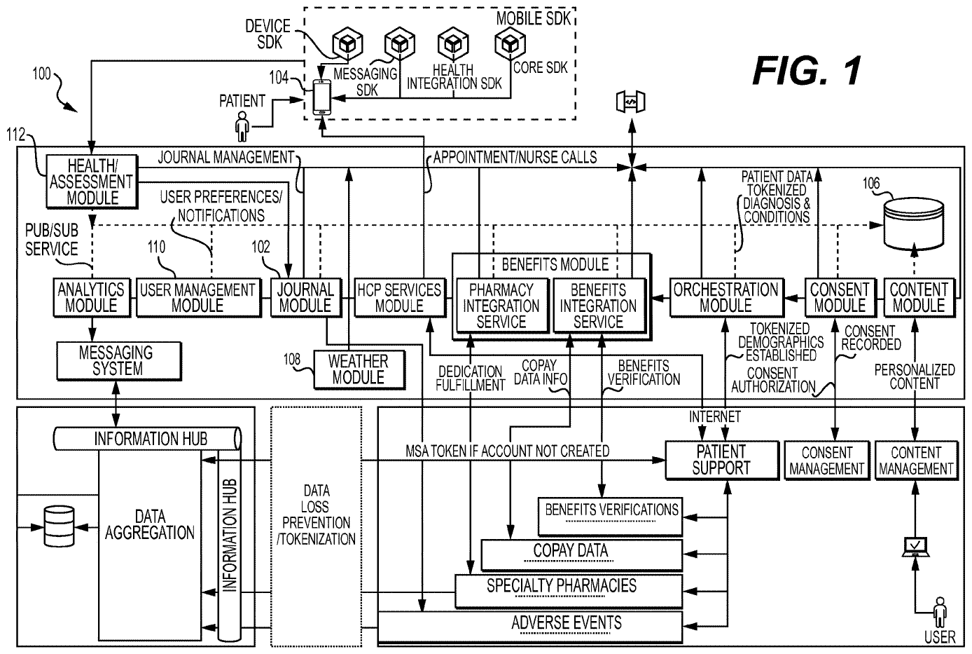

Referring to FIG. 1, schematic overview of an overall architecture 100 for a disease management and medication regimen management system is shown. The system architecture 100 illustrates a number of functional modules and the data links between these. The system architecture 100 comprises a user device 104 running an application with a number of functional modules, illustrated in the central box. The architecture 100 also illustrates how the application run by the user device 104 can interact with other service providers to enhance the information which can be provided to the user through the application. For instance, by communicating with insurance/benefit provider systems and pharmacies.

One of these functional modules is a journal module 102. The journal module 102 is configured to allow a user to maintain a journal relating to one or more medical conditions of the user. The journal may comprise a plurality of journal entries that detail episodes of the one or more medical conditions, such as symptoms, user specific health data, contextual information or the like. The journal may be used to monitor the one or more medical conditions for patterns, and to identify potential triggers for episodes of the one or more medical conditions. The journal may further maintain a record of the adherence of the user to a treatment regime, such a medicament regime. For instance, the journal entry can include a record logging a dose of medicament (e.g. a scheduled dose) that has been taken by the user, such as an injection log. The record can form a dose record including a variety of information associated with the dose administered by the user. The record can be stored as part of the journal entry or independently from the journal entry. The journal module 102 may communicate with an external service called adverse events. The journal module may transmit patient information associated with episodes of the one or more medical conditions to the adverse events service.

The functional module may further comprise a memory module, or database 106. The memory module 106 is configured to store journal entries made by the user via the journal module 102 for later retrieval and/or analysis. The memory module 106 may, in some embodiments, be lined to the cloud, and store journal entries remotely.

In some embodiments, the functional modules may further comprise a weather module 108. The weather module 108 is configured to determine and/or record weather conditions at the location of the user. The user location can be determined, for example, by a GPS capability of the user device. The weather module 108 may, for example, access an online weather forecasting system to obtain current weather conditions, previous weather conditions and/or predicted future weather conditions. The weather module 108 may provide weather conditions to the journal module 102 for inclusion in journal entries. As well as weather conditions such as general weather condition, temperature, humidity, air pressure and UV index, the weather module may additionally obtain air quality information such as pollen count, pollution index, NO₂ count and/or an air quality index.

Another of the functional modules is a user management module 110. The user management module 110 is responsible for obtaining user preferences and for generating notifications for output on the user device 104. Necessary data for the notification scheme may be stored on the memory module 106. The user management module 110 may also update the information held in the memory module 106 as the application is used.

The user management module 110 may define and help implement a notification scheme which determines when a notification should be generated and how it should be output on the user device 104. The notification scheme may have three main notification types; (i) a silent notification; (ii) a batched summary notification; and (iii) a push notification.

In some embodiments, the silent notification may be pushed into the application silently and viewable only within the application. The user may be presented with the silent notification within the application, for example on an application home screen. In some embodiments, the silent notifications are surfaced to the user when certain requirements are met and do not require any action on user's part. The batched summary notification may relate to several different reminders and other notifications. The user can interact with the batched summary notification to expand and show the individual notifications. The batch summary notification may indicate the number of notifications which are present for the user to review. The push notification may relate to a single notification and is pushed to the lock screen or home screen of the user device 104. In some embodiments, the push notifications are time-sensitive and actionable items that require the user's immediate attention. They are reserved for actions related to the user's treatment routine or as a follow up to actions that can impact the user's access to the treatment/drug.

Notifications can escalate, i.e. move up the notification types, depending on the user's action (or lack thereof). For example:
a. When the user is 5 days away from their next medicament dose, their next dosing reminder will show up as a silent notification in the application, for example underneath the carousel on the App homepage.
b. In the days leading up to the dosing, the application will push batch notifications to the user to either review instructional videos, book a call with a nurse, or review the instructions for self-administration.
c. On the day of the dosing and an hour prior to the scheduled time, the application will push a notification to the user to take their dose. If the user's medication is kept in the fridge, the notification will instruct the user to take their medicament out of the fridge. Interacting with this notification will trigger a warm-up timer, set to their medication dose.

Another of the functional modules is a Health/Assessment Module 112. The Health/Assessment Module 112 is responsible for onboarding the user and obtaining details regarding the user's medical conditions and medicaments being used to treat these. The Health/Assessment Module 112 may also gather information about the user's prescriptions and manage delivery of medicaments.

Another of the functional modules is a Health Care Professional (HCP) Services Module. The HCP Services Module is responsible for facilitating contact with a nurse, doctor or other health care professional, including scheduling reminders relating to appointments and calls, prompting the user to create reports or otherwise prepare for an upcoming appointment or call, initiating voice or video calls, providing follow-up notifications after calls or appointments and initiating a journal entry after calls or appointments. The HCP Services Module interacts with an internet-based Patient Support Program. Health care professionals may also have access to certain aspects and information held by the Patient Support Program, in order to facilitate contact with the user and monitor the user's treatment and/or disease progression.

Another of these functional modules is a Consent Module (also referred to as a consent management module) which, in conjunction with other aspects of the system, is responsible for managing consents and permissions for accessing patient specific information, such as health related data for the user. The Memory Module 106 may be configured to store user ID and role information as well as consent templates for use by the Consent Module. The Memory Module 106 may also store consent and permission information input by the user via the consent Module, including any time limits set on the consents and permissions.

The Consent Module may communicate with an external service called Consent Management service. The consent and permission information generated by the Consent Module may be stored or copied to the Consent Management service. The Consent Management service may also be responsible for sending the patient specific information to third parties when the associated consents indicate that this is permitted.

Another of the functions modules is a Content Module. The content module is responsible for receiving and recording personalised content and/or preferences set by the user. The content module may communicate with an external service called Content Management. The content management service responsible for providing patient specific content or settings from an external user, such as a healthcare professional. The content module may interact with the memory module 106 to store the personalised content and/or settings.

Another of the functional modules is a Benefits Module which, in conjunction with other aspects of the system, is responsible for managing patient access to health care. The Benefits Module may include a pharmacy integration service and/or a benefits integration service. The information generated by the benefits module may be responsible for sending patient specific information to third parties to indicate patient access to health care services. The benefits module may interact with external services, such as benefit provider systems and pharmacy services.

Another of the functional modules is an Analytics Module. The analytics module is responsible for analysing the patient information associated with the status and/or treatment of the user's medical conditions.

Another of the functional modules is a Messaging System. The Messaging System may communicate with the analytics module. For instance, if the analytics module identifies an episode or a trigger to an episode of the one or more medical conditioners of the user, the messaging system outputs a message communicating this result. The messaging system may communicate with an external service called an Information Hub. The information hub may store the data received from the messaging system. The information hub may also interact with other service providers to enhance the information which can be stored in respect of the user through the application. For instance, by communicating with the patient support program, insurance/benefit provider systems and pharmacies, adverse events services, and so on.

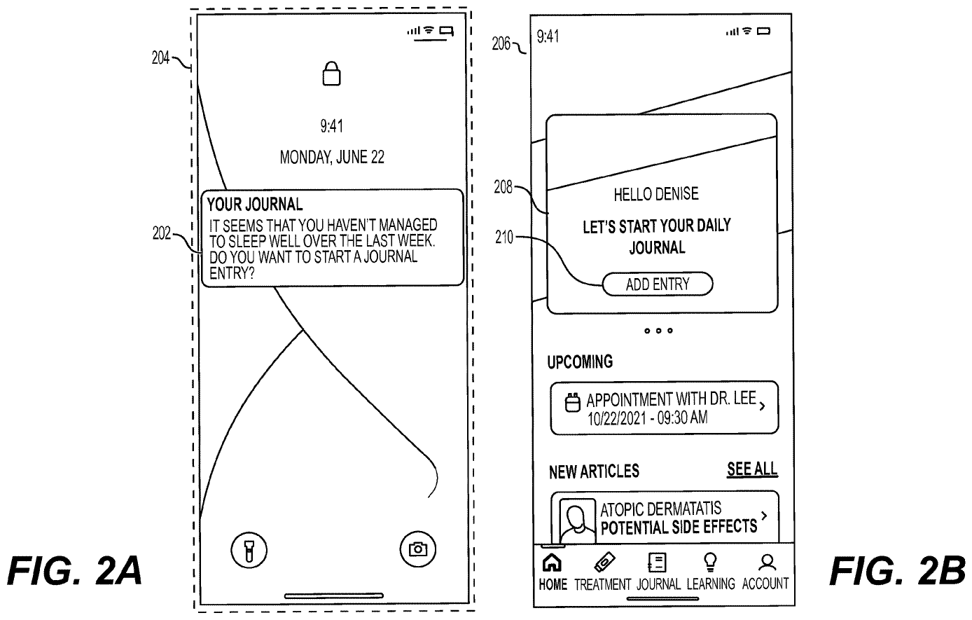

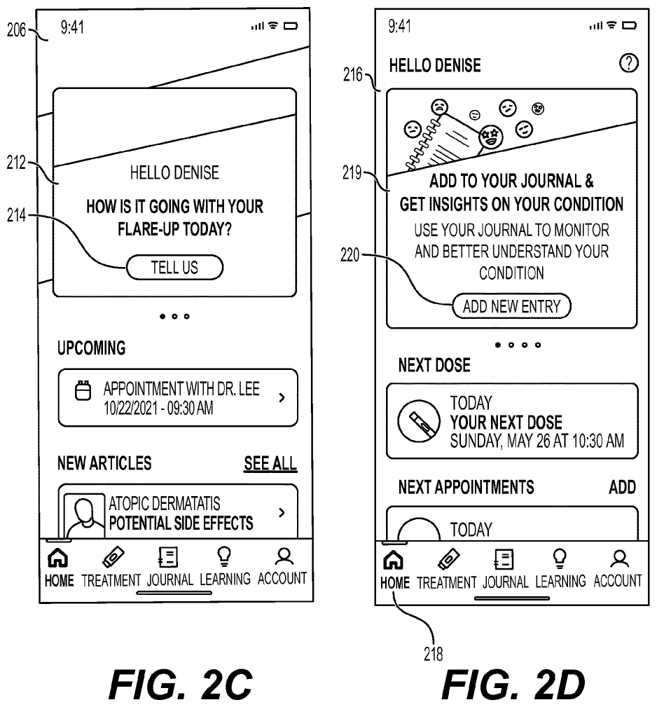

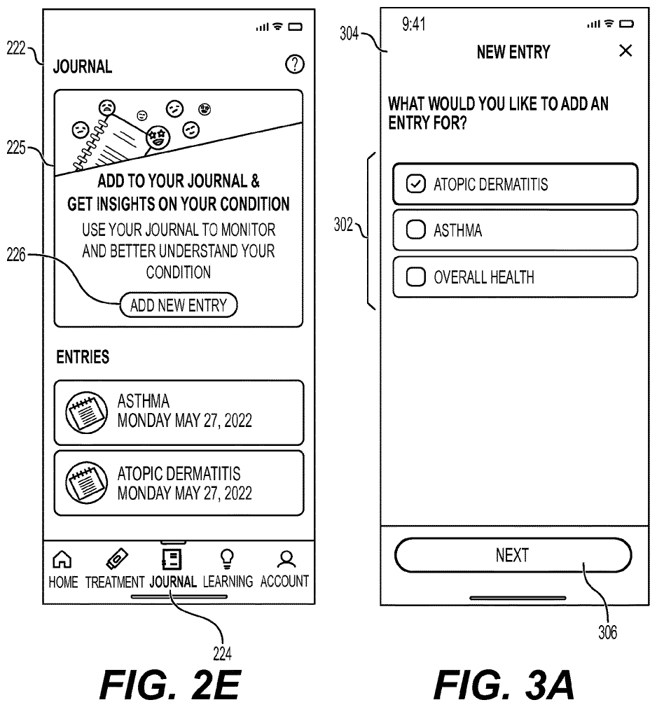

FIGS. 2a, 2b, 2c show three different ways in which the application can prompt the user to make a journal entry.

FIG. 2a shows a push notification 202 to a locked screen 204 of a user device prompting the user to make a journal entry based on detected environmental conditions or user specific health information.

The application monitors environmental conditions of the user environment, and triggers a notification 202 if the one or more environmental threshold conditions are met. The notification 202 may provide a prompt to the user to make a journal entry and, in some embodiments, provide a warning about the environmental condition that triggered the notification. The application may monitor the environmental conditions using one or more sensors present on the user device, such as a thermometer, barometer or hygrometer. Alternatively or additionally, the application may monitor the environmental conditions using environmental information from one or more other services, for example a weather application on the device or on the internet.

Examples of environmental conditions may comprise one or more of: a temperature; a humidity; an air pressure; a pollen count; a pollution index; an NO₂ count; an air quality index; a UV index; and/or a weather condition/prediction. The environmental threshold conditions may comprise upper thresholds, above which the notification is triggered and/or lower thresholds, below which the notification is triggered. For example, the environmental thresholds may comprise one or more of: an upper temperature threshold; a lower temperature threshold; an upper humidity threshold; an upper pollen count threshold; an upper NO₂ count threshold; a threshold air quality index score; and/or one or more sets of predefined weather conditions.

The application may alternatively or additionally monitor user specific health information, and trigger a notification if the one or more health condition thresholds are met. The notification may provide a prompt to the user to make a journal entry and, in some embodiments, provide a warning about the health information that triggered the notification. The user specific health information may be input by the user into the device, for example through a GUI of the device. Alternatively or additionally, the user specific health information may be collected by one or more sensors of the device, for example, a user pulse may be measured by an optical sensor on the device. Alternatively or additionally, the user specific health information may be collected from one or more external devices. For example, the application may have access to data collected by a wearable device of the user, such as a smart watch, and/or a medical device used by the user to monitor a health condition, a pulse oximeter, or a device for monitoring the user's breathing such as a peak flow meter or spirometer, or the like.

Examples of user specific health information may comprise physiological data relating to the user. For example, the user specific health information may comprise one or more of: a heart rate; a breathing rate or other breathing condition; a temperature; a blood pressure; a sleep pattern; a physical activity level; a skin condition; or the like. The user specific health information may comprise dietary information, such as calorie intake, fat intake, salt intake, vitamin/nutrient intake, or the like. The health condition thresholds may comprise upper thresholds, above which the notification is triggered and/or lower thresholds, below which the notification is triggered. The health condition threshold conditions may, for example comprise, one or more of: an upper and/or lower threshold for a heartrate; an upper/lower threshold for a blood pressure; an upper/lower temperature threshold; an upper/lower breathing rate threshold; a deviation from an established or predefined sleep pattern; or the like.

When triggered, the notification 202 may be pushed to a graphical user interface (GUI) of the device the application is running on. In the example shown, the notification 202 is pushed to the lock screen of the user device. The notification 202 may alternatively or additionally be pushed to a notification bar on the home screen of the device GUI. The notification 202 may suggest that the user make a journal entry. In some embodiments, the notification 202 may provide reasoning for why the request is being made, e.g. “It seems that you haven't been able to sleep well over the last week”. The user may select the notification 202 to open the application. The application may be opened at a home screen, or alternatively opened directly into the journal module.

FIG. 2b shows an example of a graphical user interface 206 for the application comprising in-application notification 208 prompting the user to make a journal entry. The notification may be triggered when the user starts up the application if no daily journal entry has been made that day, and prompts the user to start their daily journal entry. Alternatively or additionally, the notification may be triggered by the user entering the application via a lock screen notification, as described above in relation to FIG. 2a. The notification may comprise a GUI element, e.g. a button 210, which may be selected by the user to open the journal module at the entry for the current day. In the example shown, the GUI button for accessing the journal is marked “Add Entry”.

The GUI 206 may further comprise one or more reminders about upcoming health related events, such as appointments with a healthcare provider, scheduled medication times and/or scheduled health monitoring times (e.g. peak flow meter, pulse oximeter or spirometer check times). The reminders may comprise a link to a calendar function of the application.

Alternatively or additionally, the GUI may comprise one or more links to content (e.g. articles, webpages, videos etc.) that are relevant to the medical condition of the user. In other embodiments, one or more additional applications could track or determine a user's medical condition. For example, a vocal biomarker could provide an indication of the severity of a user's current or future asthma condition.

FIG. 2c shows an example of a graphical user interface 206 for the application comprising in-application notification 212 prompting the user to make a journal entry. The notification may be triggered when the user starts up the application in response to determining that the user is having a flare up of a medical condition. The application may determine that the user is having a flare up of a medical condition based on one or more previous journal entries to the application and/or user specific health information accessible by the application. The notification may comprise a GUI element, e.g., a button 214, which may be selected by the user to open the journal module at the entry for the current day. In other respects, the GUI may comprise the same elements as the GUI of FIG. 2b.

FIGS. 2d and 2e show an alternative view of a homepage GUI 216 and a journal page GUI 222 of the application respectively. FIG. 2d shows a homepage GUI 216. At the bottom of the page are a number of selectable tabs which are used to navigate to different areas of the application. The homepage tab 218 has been selected. The homepage GUI 216 comprises an in-application notification 219 prompting the user to make a journal entry. The notification 219 is one of several which may be displayed in this section of the GUI. The notification 219 may comprise a GUI element, e.g., a button 220, which may be selected by the user to open the journal module at the entry for the current day. In the example shown in FIG. 2d, the GUI button 220 for accessing the journal is marked “Add new entry”. The homepage GUI 216 may display other information such as the date (and optionally time) of the next scheduled dose and the date (and optionally time) of an upcoming appointment or call.

FIG. 2e shows a journal page GUI 222. At the bottom of the page are a number of selectable tabs which are used to navigate to different areas of the application. The journal page tab 224 has been selected. The journal page GUI 222 comprises an in-application notification 225 prompting the user to make a journal entry. The notification 225 is one of several which may be displayed in this section of the GUI. The notification 225 may comprise a GUI element, e.g., a button 226, which may be selected by the user to open the journal module at the entry for the current day. In the example shown in FIG. 2e, the GUI button 226 for accessing the journal is marked “Add new entry”. The journal page GUI 222 may display other information such as the date and health conditions associated with the most recent journal entries.

FIGS. 3a to 3d show the process of making a selection to create a journal entry relating to Atopic Dermatitis and the steps involved in making that entry.

In FIG. 3a, the user selects one or more health conditions 302 that the journal entry relates to from a health condition selection page 304. Selection of the one or more health conditions 302 may be performed by clicking on an associated tick-box, as shown in FIG. 3a. Alternatively or additionally, the one or more medical conditions 302 may be selected from one or more drop-down lists and/or input manually. In the example shown the medical conditions for the journal entry comprise atopic dermatitis and asthma, as well as a general “overall health” condition. In the example shown the user has selected atopic dermatitis as the relevant medical condition. Once the user has selected the one or more relevant medical conditions 302 for the journal entry, the user may select a “next” button 306 to proceed to the next part of the journal entry. The information requested by the journal entry screens that follow are determined by the selected medical condition, in this case atopic dermatitis.

In FIG. 3b, the user provides location and time details relating to an episode of the medical condition, which in this example is atopic dermatitis. The user may input a date and/or time 308 that the atopic dermatitis episode occurred. The date may be selected from a calendar. The user may further indicate a location 310 that that event occurred, e.g. by inputting an address, selecting from a list of predetermined location (for example, “home”, “work” or the like) and/or selecting a location on a map, or selecting a location proposed by the user device's location service (e.g., GPS). In the example shown, the user has indicated that the episode occurred on Mar. 22, 2021, at a time of 9.45 am and at a location of 1255 Rarlton Road, New York. In some embodiments, the location box is omitted. The application may display a similar GUI asking the user to “Select the date to add an entry for” and providing the date and time boxes only.

In some embodiments, the GUI comprises a progress indicator 312 that indicates how far the user has progressed in completing the journal entry. In the example shown, the progress indicator 312 is located at the top of the GUI, and comprises a sequence of numbered icons, each corresponding to a page of the journal entry. The current page of the journal entry is highlighted in the progress indicator 312. As the user completes each page of the journal entry, the progress indicator 312 may mark the corresponding icon with an indication that it has been completed, e.g. a tick. In some embodiments, the icons in the progress indicator 312 may additionally be used as shortcuts to the corresponding journal page. The progress indicator 312 may alternatively be in the form of a progress bar and/or a numerical progress indicator (e.g. a percentage of completion).

Once the user has input the date and location details of the episode, the user may click on a “next” button 306 to proceed to the next part of the journal entry. Alternatively, to return to the health condition selection page, the user may select a back button 314.

In FIG. 3c, the user is provided with one or more queries 316 regarding the health condition. In the example shown, the queries 316 relate to atopic dermatitis. The queries 316 may answered by selecting a symbolic score (e.g. a face) or a numerical score (e.g. a number between zero and four or between zero and five) from a set of potential scores. In the example shown, the selection is performed by selecting one of the displayed numbers, though other methods of inputting the scores may alternatively be used, e.g. a drop-down box, a text input, a slider or the like. In some embodiments, one or more of the queries may be in the form of a “yes/no” or “true/false” statement.

The queries 316 may relate to aspects of the health condition (e.g., for atopic dermatitis, “Skin lesion level”, “What is your level of itchiness?”, “How is the level of pain?”, “Quality of life impact”, “Sleep disturbance impact” etc.) or to the general condition of the user with respect to the health condition (e.g., “How are you feeling now?”).

Once the user has input answers to the queries 316, the user may click on a “next” button 306 to proceed to the next part of the journal entry. Alternatively, to return to the location and time details page, the user may select a back button 314.

In FIG. 3d, the user is asked if they wish to add any further details about their symptoms. This page may comprise a first section 315 for indicating symptoms which have gotten better and a second section 317 for indicating symptoms which have gotten worse. The list of symptoms in the first and second section will depend on the health condition to which the journal entry relates. In the case of Atopic Dermatitis they may be “Skin lesions”, “Itchiness” and “Sleep disturbance”. Once the user has input any symptom details that they wish to, the user may click on a “next” button 306 to proceed to the next part of the journal entry. Alternatively, to return to the query page, the user may select a back button 314.

The next part of the journal entry may comprise a photo entry page (not shown). This page gives the user the option to take photographs (or videos) of their body to add to the journal. Upon selecting a displayed option to add a photo or video, the application may display options to open a camera application on the user device to capture a new photo or a photo gallery application on the user device to choose an existing photo.

In FIG. 3e, the user inputs one or more body locations 318 at which the health condition, e.g. atopic dermatitis, is occurring. Such information may only be relevant to certain health conditions, and as such, this journal page may not be present in all situations. The user may indicate one or more locations 318 at which the health condition is occurring/an episode of the health condition is occurring. If the user has previously indicated that the health condition is occurring at one or more body locations 318, the user may indicate which locations are getting worse. In the example shown, the user may indicate one or more locations 318 by selecting one or more pre-defined location on a model of the human body. The model may be a rotatable model, allowing the user to select locations on the front, back and sides of the body. Other methods of inputting the body locations may alternatively be used, such as selecting the locations from a list or inputting them manually.

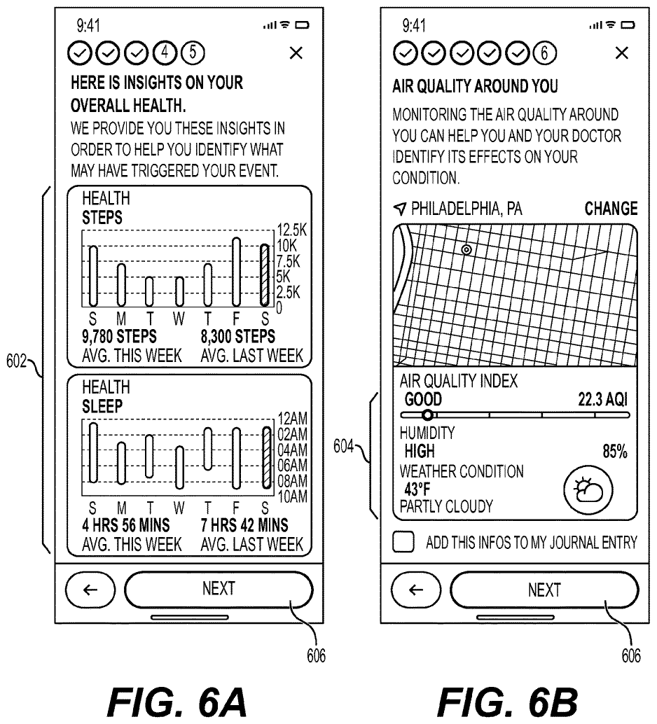

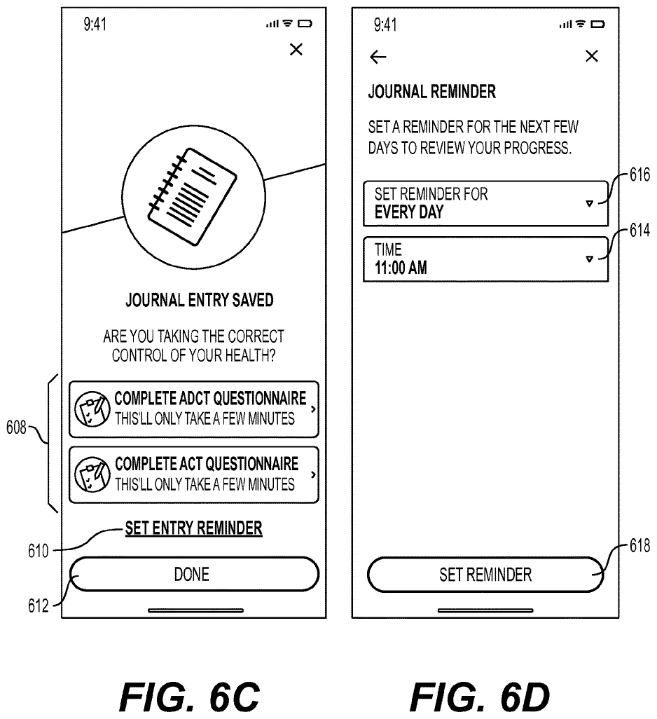

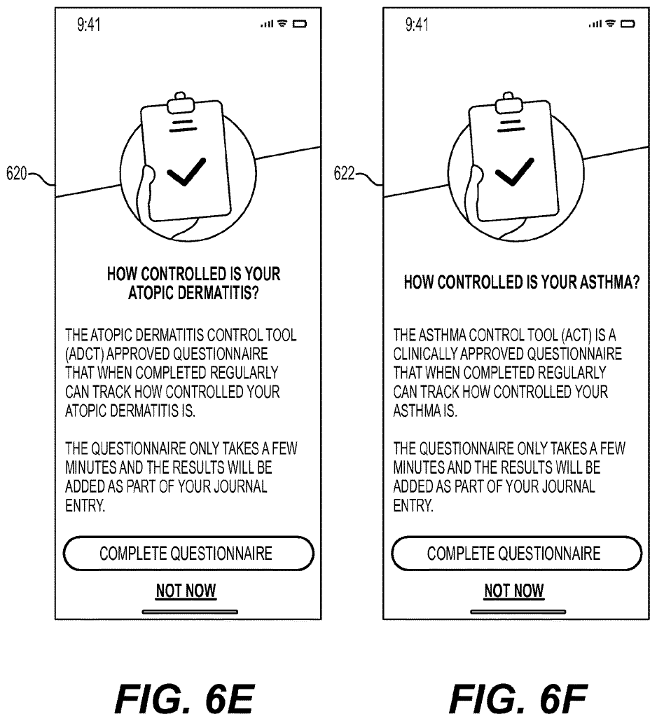

Once the user has input the one or more body locations 318, the user may click on a “next” button 306 to proceed to the next part of the journal entry. If no other medical conditions were selected in FIG. 3a, the application proceeds to FIG. 6b. Otherwise, the method proceeds to

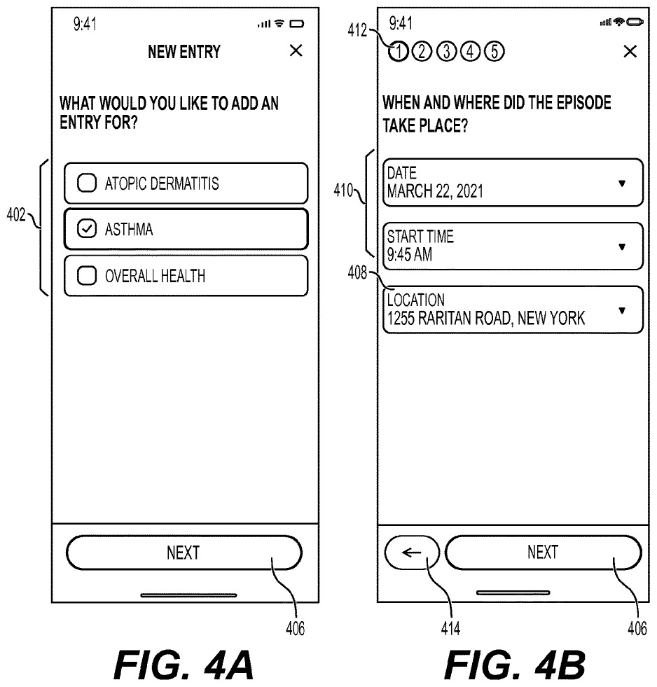

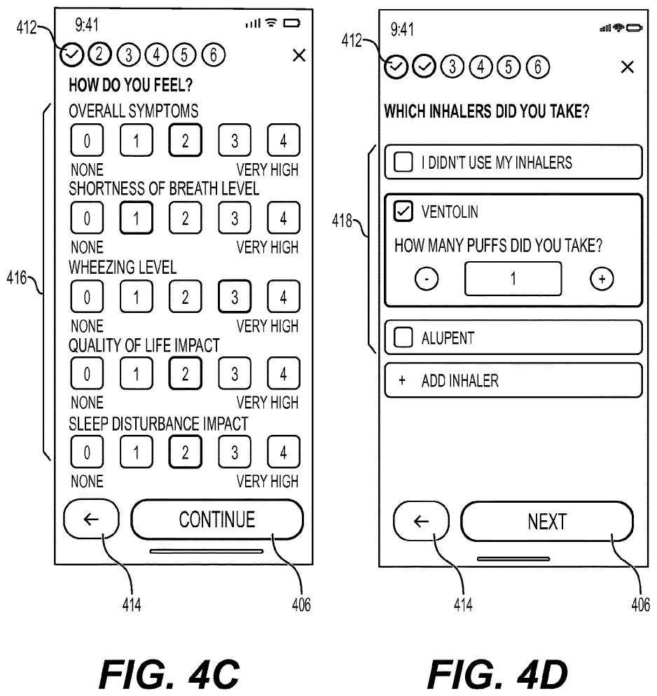

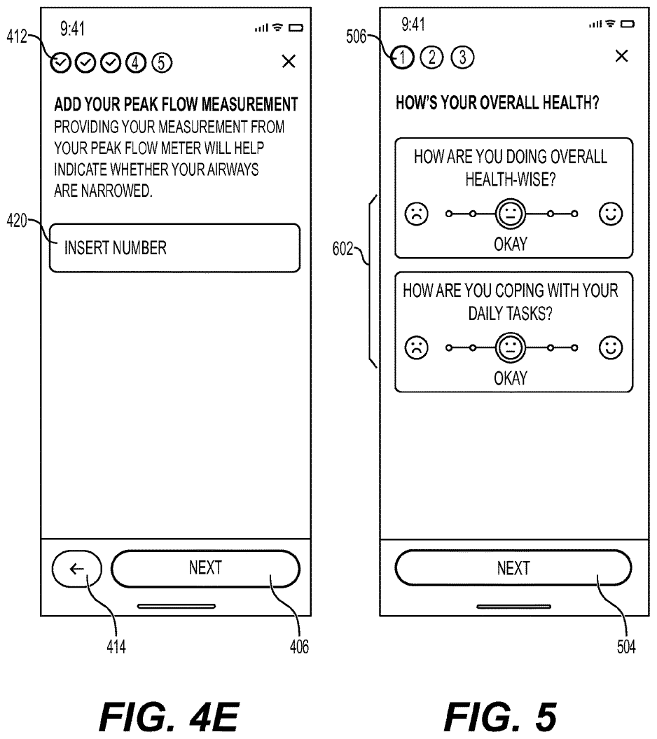

FIG. 4 or FIG. 5, depending on the other conditions(s) selected. Alternatively, to return to the symptom details page or photo entry page, the user may select a back button 314.

FIGS. 4a to 4e show the process of making a selection to create a journal entry relating to Asthma and the steps involved in making that entry.

In FIG. 4a, the user selects one or more health conditions 402 that the journal entry relates to from a health condition selection page, as described above in relation to FIG. 3a. In the example shown the user has selected asthma as the relevant medical condition. Once the user has selected the one or more relevant medical conditions 402 for the journal entry, the user may click on a “next” button 406 to proceed to the next part of the journal entry. The information requested by the journal entry screens that follow are determined by the selected medical condition, in this case asthma.

In FIG. 4b, the user provides location 408 and time 412 details relating to an episode of the medical condition, which in this example is asthma. The user inputs the location 408 and time details 410 as described above in relation to FIG. 3b. Once the user has input the date and location details of the episode, the user may click on a “next” button 406 to proceed to the next part of the journal entry. Alternatively, to return to the health condition selection page, the user may select a back button 414. In some embodiments, the location box is omitted. The application may display a similar GUI asking the user to “Select the date to add an entry for” and providing the date and time boxes only.

In some embodiments, the GUI comprises a progress indicator 412 that indicates how far the user has progressed in completing the journal entry, as described above in relation to FIG. 3b.

In FIG. 4c, the user is provided with one or more queries 416 regarding the health condition. In the example shown, the queries relate to asthma. The user may input the answers to the queries 416 in the manner described above in relation to FIG. 3c. The queries 416 may relate to aspects of the health condition (e.g., for asthma, “Overall symptoms” “What level is your shortness of breath?”, “Wheezing level”, “Quality of life impact”, “Sleep disturbance impact” etc.) or to the general condition of the user with respect to the health condition (e.g. “How are you feeling now?”). Once the user has input the query responses, the user may click on a “next” button 406 to proceed to the next part of the journal entry. Alternatively, to return to the location and time page, the user may select a back button 414.

In FIG. 4d, the user is provided with one or more queries relating to medication for the health condition 418. The medication queries 418 may comprise identifying a medicament and dose administered by the user to treat the condition. A list of medicaments for the condition may be provided, alongside GUI elements for inputting the dose administered. In some embodiments, the list of medicaments may be restricted to medicaments prescribed to the user; in other embodiments the list may comprise a more general list of medicaments available for treatment of the condition. The user may add additional medications to the list manually. The GUI may also provide the option to indicate that no medicament has been taken.

In the example shown, the user is provided the option of selecting one of two asthma inhalers, Ventolin or Alupent, or indicating that an inhaler has not been used. The user also has the option to add additional inhalers. For each inhaler, the user can indicate a number of puffs of the inhaler taken.

Once the user has input the medication responses, the user may click on a “next” button 406 to proceed to the next part of the journal entry. Alternatively, to return to the query page, the user may select a back button 414.

In FIG. 4e, the user inputs one or more physiological measurements 420 relevant to the medical condition. The user my input the physiological measurements 420 manually, as shown, or read then in from an external device, for example using a network connection or RFID reader of the user device. In the example shown, the user is asked to input a peak flow measurement, which is indicative of whether the airways of the user have narrowed.

Once the user has input the one or more physiological measurements 420, the user may click on a “next” button 406 to proceed to the next part of the journal entry. If no other medical conditions were selected in FIG. 3a, the application proceeds to FIG. 6. Otherwise, the method may proceed to FIG. 5, depending on the other conditions(s) selected. Alternatively, to return to the medication page, the user may select a back button 414.

In addition to the screens described above, for Asthma the application may also display a list of symptoms, in a similar way to that shown in FIG. 3d. The user may then indicate which symptoms are getting better or getting worse. For Asthma, an exemplary list of symptoms may be “Shortness of breath”, “Wheezing” and “Sleep disturbance”. The application may also offer the user the option to capture or upload photos or videos, or make a sound recording. For certain health conditions such as Asthma, a video or sound recording may be informative as to the severity of the user's symptoms.

FIG. 5 shows the process of making a selection to create a journal entry relating to Overall Health and the single page with the entry options. The user is presented with one or more queries 602 relating to the overall health of the user. The queries may answered by selecting a symbolic score (e.g. a face) or a numerical score (e.g. a number between zero and five) from a set of potential scores, as described in relation to FIG. 3c. In the example shown, the general health queries are “How are you doing overall health-wise?” and “How are you coping with your daily tasks?”, though many other examples are possible.

In some embodiments, the GUI comprises a progress indicator 506 that indicates how far the user has progressed in completing the journal entry, as described above in relation to FIG. 3b.

Once the user has input the query answers, the user may click on a “next” button 504 to proceed to the next part of the journal entry. The application then proceeds to FIG. 6.

FIGS. 6a to 6d show examples of screens which may be displayed after FIGS. 3d, 4e and/or 5 in their respective processes showing insights on overall health and environmental data.

In FIG. 6a, the user is presented with one or more overall health insights 602. The insights 602 are provided to assist the user in identifying causes/triggers of the episode/flare up of the health condition. The health insights 602 may be based on physiological data collected from the user and/or responses to the queries provided in the FIGS. 3-5. In the example shown, the user is presented with a step count for each of the preceding seven days, which may be indicative of an amount of activity performed by the user. The step count is presented in the form of a bar chart. The user is also provided with a sleep indicator, indicating the times at which the user has been able to sleep in the last seven days. A record of the information presented in the health insights may be recorded and associated with the journal entry. Once the user has viewed the health insights, the user may click on a “next” button 606 to proceed to the next part of the journal entry.

In FIG. 6b, the user is presented with one or more environmental conditions 604 at or near the time of the episode of the medical condition. These may assist the user in identifying the trigger of the episode/flare-up of the medical condition. The environmental conditions 604 may, for example comprise atmospheric conditions, e.g., an air quality index, a NO₂ value, a pollen count, a humidity, a temperature and/or a weather condition. Once the user has viewed the environmental conditions 604, the user may click on a “next” button 606 to proceed to the next part of the journal entry. The screen presenting the environmental conditions 604 may also comprise an option (for example a checkbox) to add the environmental condition information to the user's journal entry. In this manner, the environmental condition information can be associated with the user's symptoms and used to better inform the user of environmental conditions which may affect them, provide more timely notifications to make future journal entries, or provide a warning to the user about current or predicted environmental conditions in their location, or a location they intent to travel to.

In some implementations, the application (or remote systems receiving information from the application) may automatically determine one or more risk factors for the user's medical condition by correlating the symptoms of the user's medical condition with the associated external/environmental conditions and/or other associated user-specific health information, such as sleep pattern or duration or physical activity. Using data from a plurality of journal entries, the application may identify recurring external/environmental conditions (or user-specific health information) that occur in conjunction with episodes of a medical condition of the user. In some embodiments, the identification may be based on the relative frequency that a set of external/environmental conditions (or user-specific health information) occur within a predefined time period of an episode. Alternatively, one or more medical condition-specific rules may be applied to identify the one or more risk factors from the environmental or health information.

In FIG. 6c, the user is informed that the journal entry has been saved to the memory of the device (or the cloud, if cloud features are enabled). The user may select one or more questionnaires 608 to complete. The questionnaires may comprise a series of further questions about the user's health condition, and may facilitate free-text input. A disease control score may be presented to the user at the end of the questionnaire along with any suggested actions or links to further information and support. The user may also be presented with the option to set a reminder 610 to make an journal entry; if the user selected this option, the user is presented with the screen shown in FIG. 6c. Alternatively, the user may select a “Done” GUI element 612 to complete the journal entry process.

FIG. 6d shows the screen which is displayed if the user selects the set entry reminder text 610 on FIG. 6c. Via this screen, a user may set up a recurring alarm to remind them to make a journal entry. The user may select a reminder time 614 and a recurrence rate 616 (e.g. every day, every week etc.). Once the alarm time 614 and recurrence 616 are selected, the user may finalize the alarm by selecting a “set reminder” GUI element 618, e.g. button. The alarm may push notifications to the user device, for example as described in relation to FIG. 2a, when the alarm time is reached.

FIG. 6e shows an alternative screen 620 which may be displayed after a primary journal entry relating to Atopic Dermatitis is complete. This screen 620 gives the user the option to complete an Atopic Dermatitis Control Tool (ADCT) questionnaire. The screen 620 has options to begin the questionnaire or to skip the questionnaire. FIG. 6f shows an alternative screen 622 which may be displayed after a primary journal entry relating to Asthma is complete. This screen 622 gives the user the option to complete an Asthma Control Tool (ACT) questionnaire. The screen 620 has options to begin the questionnaire or to skip the questionnaire.

While FIG. 3a and FIG. 4a show the selection of single health condition, the user may at this point in the journal entry process select two or more health conditions. For example, the user may select both Atopic Dermatitis and Asthma. The application would then go through the process of creating a journal entry relating to Atopic Dermatitis followed sequentially by the journal entry process relating to Asthma. For example, the application may go through the journal entry process as described in relation to FIGS. 3b to 3d followed by the journal entry process as described in relation to FIGS. 4b to 4e. If “Overall health” was also selected, then the process as described in relation to FIG. 5 is then performed. Other permutations are possible depending on the range of health conditions being managed by the application. After the journal entry process for all selected health conditions is completed, the application may show the overall health insights screen of FIG. 6a and the environmental conditions screen of FIG. 6b, as previously described.

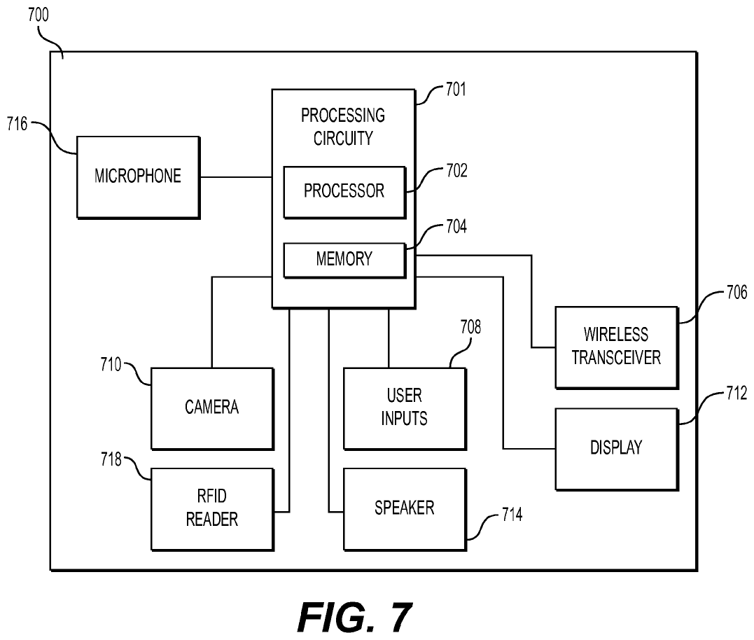

FIG. 7 illustrates schematically a user device 700 according to some embodiments. The user device 700 may be a mobile phone or tablet computer. The user device 700 is an example of a wireless communication device. The user device may alternatively be referred to as a communication device, computer, computing device or mobile device and is not necessarily associated with a single user. The user device 700 is configured to communicate wirelessly with a communications network using a wireless communication protocol, for example but not exclusively 3GPP LTE and/or New Radio (NR) or Wi-Fi (IEEE 802.11). The user device 700 may also be configured to communicate using Bluetooth, NFC, Zigbee, Ultra Wideband, IrDa or similar. The communications network may comprise one or more network nodes. The communications network may further be connected via a core network and/or an intermediate network to a host computer (not shown) which may be embodied in the hardware and/or software of a standalone server, a cloud-implemented server or a distributed server. The user device 700 and the host computer may be configured to communicate data.

The user device 700 may comprise hardware that includes processing circuitry 701. The processing circuitry 701 may comprise a processor 702 and a memory 704. The user device 700 may also comprise a wireless transceiver 706, user inputs 708, a display 712, a camera 710, a microphone 716, and RFID reader 718 and a speaker 714. The wireless transceiver 706 may be configured to set up and maintain a wireless connection to a network node. The wireless transceiver 706 may comprise one or more radio transmitters and one or more radio receivers. The display may be a touch sensitive display and may be based on capacitive or resistive sensing technology. The processing circuitry 701 may for instance include a microprocessor, a Digital Signal Processor (DSP), Application Specific Integrated Circuit (ASIC), Field Programmable Gate Array (FPGA) or the like. The processor 702 may be configured to read and/or write from the memory 704. The memory 704 may comprise a volatile and/or nonvolatile memory, for example a cache, RAM (Random Access Memory) and/or ROM (Read-Only Memory) etc.

The user device 700 may comprise software which is stored, for example, in memory 704. The software may be executable by the processing circuitry 701. The software may include an application. In some embodiments, the host computer may communicate with the application. The application may request data from the host computer and/or provide user data to the host computer.

The processing circuitry 701 may be configured to perform or cause to be performed any of the methods described herein. In some embodiments, the software/program may include instructions that, when executed by the processing circuitry 701 cause the processing circuitry 701 to perform the methods described herein.

The memory 704 may comprise both a program memory storing program code (e.g. software or firmware) and main memory storing data. The processing circuitry 701 may be configured to execute the program code stored in the program memory and to read, write and delete data from the main memory. In some embodiments, the program code may be an application which can be downloaded and installed on the user device 700. The application may be a disease and treatment management and tracking tool for use by patients. The program memory may for instance be a Read-Only Memory (ROM), and the main memory may for instance be a Random Access Memory (RAM).

The user device 700 comprises one or more user inputs 708, for example a touchscreen, keypad or keyboard, accelerometer or gyroscope, mouse or microphone 716 for receiving voice commands. User device 700 may also comprise a camera 710 configured to capture images of a user and images of labels, codes and the like visible on the medicament administration devices, packaging or storage solutions. The user device 700 may be configured to scan medicament administration devices (such as an injection device or inhaler) using a scanning device. The scanning device may refer to either the camera 710 or RFID reader 718. The term “scanning” as used in relation to the user device 700 may refer to use of either of these components to read information provided externally or internally on medicament administration devices.

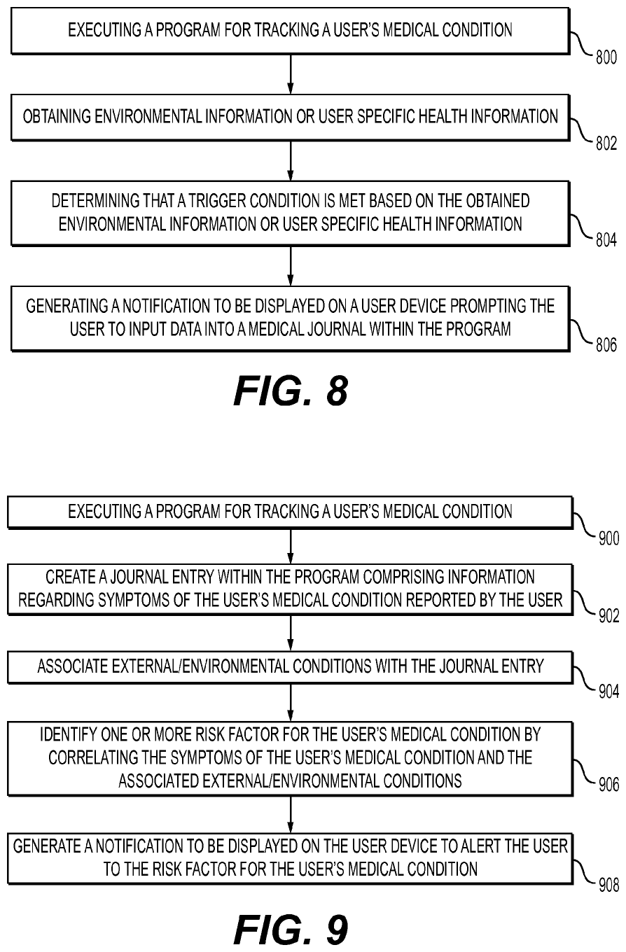

FIG. 8 shows a flow chart of an example method for triggering a journal notification. The method may be performed by computing apparatus/systems, such as those described in relation to FIG. 7. The computing apparatus/system may be a user device

At operation 800, a program (also referred to as an application) for tracking a medical condition of a user is executed on the computing apparatus/system.

At operation 802, environmental data and/or user specific health information is obtained by the computing apparatus/system. The obtained environmental information and/or user specific health information is indicative of a possible deterioration of the user's medical condition.

The environmental data may comprise current environment data relating to a current state of the environment of the user. The computer may obtain the environmental information using one or more sensors forming part of the user device, e.g. a thermometer, a barometer and/or a hygrometer. Alternatively or additionally, the environmental information may be obtained from a weather application running on the user device. The environmental data may, for example, comprise weather data. The weather data may comprise one or more of: a temperature; an air pressure; a humidity; a UV index; and/or a weather state (e.g. “raining”, “sunny”, “stormy” etc.). The environmental data may alternative or additionally comprise one or more measures of air quality, such as an air quality index, an NO₂ level, a pollen count or the like.

The user specific health information may comprise an indication of a possible deterioration of the user's medical condition. The user specific health information may comprise one more physiological measurements taken from the user. The physiological measurements may, for example, comprise one or more of: a heart rate; a blood pressure; a blood glucose level; a body temperature; a blood oxygen level or the like. The user specific health information may alternatively or additionally comprise one or more activity measurements, e.g. a step count, a sleep pattern, a distance travelled or the like. The user specific health information may alternatively or additionally comprise one or more dietary measurements, e.g. a calorie intake, fat intake and/or salt intake.

One or more sets of user specific health information may be input to the computing apparatus/system manually by the user. Alternatively or additionally, one or more sets of user specific health information may be captured by the computing apparatus/system using one or more on-device sensors, such as an optical sensor. Alternatively or additionally, one or more sets of user specific health information may be input to the computing apparatus/system from an external device, e.g. a smartwatch, a step-meter, a glucose meter or a pulse oximeter, via a network interface, e.g. via Wi-Fi, Bluetooth and/or an RFID tag reader.

At operation 804, the computing apparatus/system determines whether one or more trigger conditions are been satisfied based on the obtained environmental information and/or user specific health information. The trigger conditions may be based on one or more thresholds for the environmental data and/or user specific health information.

The one or more trigger conditions may comprise one or more of: an upper temperature threshold; a lower temperature threshold; an upper humidity threshold; an upper pollen count threshold; an upper NO₂ count threshold; a threshold air quality index score; and/or one or more sets of predefined weather conditions. The one or more trigger conditions may alternatively or additionally comprise one or more of: an upper and/or lower threshold for a heartrate; an upper/lower threshold for a blood pressure, a lower threshold for a blood oxygen level, an upper/lower temperature threshold, an upper/lower breathing rate threshold, a lower threshold for a number of steps taken in a specific time period and/or a deviation from an established or predefined sleep pattern.

At operation 806, in response to determining that a trigger condition is met, the computing apparatus/system generates a notification to be displayed on a user device prompting the user to input data into a medical journal within the program. The notification may provide information relating to the trigger that caused the notification to be generated. The notification may, for example, be provided as a push notification to a locked screen of the user device.

The user may respond to the notification by creating a journal entry within the program comprising information regarding symptoms of the user's medical condition reported by the user, for example as described above in relation to FIGS. 3-6. The journal entry may relate to one or more medical conditions, e.g. a plurality of medical conditions.

The application may compare the information regarding symptoms of the user's medical condition reported by the user in the journal entry with contemporary environmental information or user specific health information to identify one or more correlations. Based on the identified one or more correlations, the system may identify that current environmental information or user specific health information is indicative of a risk factor for the user's medical condition. In response, the system may generate a notification to be displayed on the user device to alert the user to the current environmental information or user specific health information and/or to the risk factor.

FIG. 9 shows a flow chart of an example method for identifying a risk factor for a user's medical condition.

At operation 900 a program (also referred to as an application) for tracking a medical condition of a user is executed on the computing apparatus/system. The method may be performed by computing apparatus/systems, such as those described in relation to FIG. 7.

At operation 902 a journal entry within the program comprising information regarding symptoms of the user's medical condition reported by the user. The journal entry may, for example, be created using any of the methods described in relation to FIGS. 3-6.

At operation 904, one or more external/environmental conditions are associated with the journal entry, for example as described in relation to FIG. 2.

At operation 906, one or more risk factor for the user's medical condition are identified by correlating the symptoms of the user's medical condition and the associated external/environmental conditions. The correlation may be based on a frequency of the external/environmental conditions occurring within a predefined time period of a flare up/episode of the medical condition. The correlation may be determined based on a plurality of journal entries.

At operation 908, a notification to be displayed on the user device to alert the user to the risk factor for the user's medical condition is generated.

In some embodiments, the method may further comprise predicting when one or more further flare ups/episodes may occur based on identifying that one or more current external/environmental conditions correspond to an identified risk factor. When it is determined that one or more current external/environmental conditions correspond to an identified risk factor, a warning notification may be provided to the user via a GUI of the computing apparatus/system. The warning may provide suggested preventative techniques for avoiding a flare up/episode under the current conditions. To this end, the method may obtain information regarding new environmental conditions and/or new user specific health information. The system then determines that the new environmental conditions and/or new user specific health information is correlated with an identified risk factor for the user's medical condition. The system then generates a notification to be displayed on the user device to alert the user to the new environmental information or new user specific health information and/or to the risk factor. The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about 4 °C to about 4 °C). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body. The drugs or medicaments contained in the drug delivery devices as described herein can be used for the treatment and/or prophylaxis of many different types of medical disorders.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6R mAb (e.g., Sarilumab), and anti IL-4R mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014(E). As described in ISO 11608-1: 2014(E), needle-based injection systems may be broadly distinguished into multi-dose container systems and single-dose (with partial or full evacuation) container systems. The container may be a replaceable container or an integrated non-replaceable container.

As further described in ISO 11608-1: 2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1-29. (canceled)

30. One or more non-transitory computer-readable storage media storing instructions that, when executed by a computer, cause the computer to:
execute a program for tracking a medical condition of a user;
obtain environmental information or user specific health information, wherein the obtained environmental information or user-specific health information is indicative of a possible exacerbation of the medical condition of the user;
determine that a trigger condition is met based on the obtained environmental information or user specific health information; and
in response to determining that the trigger condition is met, generate a notification to be displayed on a user device prompting the user to input data into a medical journal within the program.

31. The one or more non-transitory computer-readable storage media of claim 30, wherein the program is configured to track a plurality of different medical conditions of the user.

32. The one or more non-transitory computer-readable storage media of claim 30, wherein the computer is part of the user device and wherein the instructions are further configured to cause the computer to perform at least one of the following:
obtaining the environmental information using one or more first sensors forming part of the user device;
obtaining the environmental information from a weather application running on the user device;
obtaining the user specific health information from one or more second sensors of the user device; or
displaying the generated notification on the user device as a push notification to a locked screen of the user device.

33. The one or more non-transitory computer-readable storage media of claim 32, wherein the one or more first sensors comprise one or more of a thermometer, a barometer, or a hygrometer.

34. The one or more non-transitory computer-readable storage media of claim 30, wherein the environmental information comprises one or more of: a temperature; a humidity; an air pressure; a pollen count; a pollution index; an NO₂ count; an air quality index; a UV index; a weather condition; or a weather prediction.

35. The one or more non-transitory computer-readable storage media of claim 30, wherein the trigger condition comprises one or more of: an upper temperature threshold; a lower temperature threshold; an upper humidity threshold; an upper pollen count threshold; an upper NO₂ count threshold; a threshold air quality index score; or one or more sets of predefined weather conditions.

36. The one or more non-transitory computer-readable storage media of claim 32, wherein the one or more second sensors comprise an optical sensor.

37. The one or more non-transitory computer-readable storage media of claim 30, wherein the instructions are further configured to cause the computer to obtain the user specific health information from one or more external devices.

38. The one or more non-transitory computer-readable storage media of claim 37, wherein the one or more external devices comprises a wearable device selected from a list of devices comprising a smart watch, a step meter, a glucose meter, and a pulse oximeter.

39. The one or more non-transitory computer-readable storage media of claim 30, wherein the user specific health information comprises one or more of: a heart rate, a breathing rate, a temperature, a blood pressure, a blood oxygen level, a number of steps taken in a specific time period, an amount of sleep, or a sleep pattern.

40. The one or more non-transitory computer-readable storage media of claim 39, wherein the trigger condition comprises one or more of: an upper and/or lower threshold for a heartrate, an upper/lower threshold for a blood pressure, a lower threshold for a blood oxygen level, an upper/lower temperature threshold, an upper/lower breathing rate threshold, a lower threshold for a number of steps taken in a specific time period, or a deviation from an established or predefined sleep pattern.

41. The one or more non-transitory computer-readable storage media of claim 30, wherein the user specific health information comprises one or more of: calorie intake, fat intake, or salt intake.

42. The one or more non-transitory computer-readable storage media of claim 30, wherein the generated notification provides a warning about the environmental information or the user specific health information that triggered the generation of the notification.

43. The one or more non-transitory computer-readable storage media of claim 30, wherein the instructions are further configured to cause the computer to create a journal entry within the program comprising information regarding symptoms of the user's medical condition reported by the user.

44. The one or more non-transitory computer-readable storage media of claim 43, wherein the information regarding symptoms of the user's medical condition comprises information regarding symptoms of a plurality of different medical conditions.

45. The one or more non-transitory computer-readable storage media of claim 43, wherein the instructions are further configured to cause the computer to:
compare the information regarding symptoms of the user's medical condition reported by the user with contemporary environmental information or user specific health information to identify one or more correlations;
based on the identified one or more correlations, identify that current environmental information or user specific health information is indicative of a risk factor for the user's medical condition; and
in response, generate a notification to be displayed on the user device to alert the user to the current environmental information or user specific health information and/or to the risk factor.

46. A computer-implemented method comprising:
executing a program for tracking a user's medical condition;
obtaining environmental information or user specific health information;
determining that a trigger condition is met based on the obtained environmental information or user specific health information; and
in response to determining that the trigger condition is met, generating a notification to be displayed on a user device prompting the user to input data into a medical journal within the program.

47. The computer-implemented method of claim 46, further comprising at least one of the following:
obtaining the environmental information using one or more first sensors forming part of the user device;
obtaining the environmental information from a weather application running on the user device;
obtaining the user specific health information from one or more second sensors of the user device; or
displaying the generated notification on the user device as a push notification to a locked screen of the user device.

48. A user device comprising one or more processors and one or more storage devices storing instructions that, when executed by the one or more processors, cause the one or more processors to:
execute a program for tracking a user's medical condition;
obtain environmental information or user specific health information;
determine that a trigger condition is met based on the obtained environmental information or user specific health information; and
in response to determining that the trigger condition is met, generate a notification to be displayed on a user device prompting the user to input data into a medical journal within the program.

49. The user device of claim 48, wherein the instructions, when executed by the one or more processors, further cause the one or more processors to perform at least one of the following:
obtaining the environmental information using one or more first sensors forming part of the user device;
obtaining the environmental information from a weather application running on the user device;
obtaining the user specific health information from one or more second sensors of the user device; or
displaying the generated notification on the user device as a push notification to a locked screen of the user device.
